(19) United States
(12) Patent Application Publication
(10) Pub. No.: US 2016/0354596 A1
(43) Pub. Date: Dec. 8, 2016

(54) ACTIVE-AGENT INSERTS FOR CONNECTORS AND RELATED METHODS

(71) Applicant: ATTWILL MEDICAL SOLUTIONS STERIFLOW L.P., Salt Lake City, UT (US)

(72) Inventor: Attilio DiFiore, West Jordan (JP)

(21) Appl. No.: 15/111,971

(22) PCT Filed: Jan. 16, 2015
(86) PCT No.: PCT/US15/11722

§ 371 Date: Jul. 15, 2016

Related U.S. Application Data
(60) Provisional application No. 61/928,378, filed on Jan. 16, 2014.

(51) Int. Cl. A61M 39/16; A61M 25/00; A61M 39/12; A61M 39/18; A61M 39/20

(52) U.S. Cl. CPC A61M 39/162; A61M 39/18; A61M 39/20; A61M 39/12; A61M 25/0017; A61M 2205/0205; A61M 2039/1077

(57) ABSTRACT

A method of tuning the release of an active agent from a cartridge disposed in a catheter and related cartridges are disclosed herein. In one embodiment, a method of tuning the release of an active agent from a cartridge disposed in a catheter convector is provided. The method can include establishing a desired release profile for an active agent to be delivered into a catheter, and disposing a cartridge having the active agent associated therewith into a fluid pathway of a catheter connector of the catheter. Thus, when an aqueous solution contacts and passes by the cartridge, the active agent is released from the cartridge at the desired release profile.

### BACKGROUND

Access devices, such as catheters, are subject to causing infection and can have biofouling issues. Infection and intervention rates on access catheters can vary based upon the type of catheter and duration of placement. Infection of long-term blood access devices in particular can be associated with blood stream infections that can lead to patient death. Interventions designed to rescue an access from removal due to clotting or biofouling often requires that the patient undergo a revision treatment where the access device is removed and replaced. In some cases, it is necessary to sacrifice the compromised access location and place the device in a secondary location in the patient. Extending the patency of access devices can improve the viability of long-term placement and reduce the need for subjecting patients to the expense and trauma of unnecessary additional intervention procedures.

### BRIEF DESCRIPTION OF THE DRAWINGS

FIG. 1 is a schematic diagram of an example insert in accordance with the present technology;

FIG. 2A is a schematic diagram of the example insert of FIG. 1 prior to coupling with a syringe and a catheter fitting;

FIG. 2B is a schematic diagram of the example insert of FIG. 1 after coupling with a syringe and a catheter fitting;

FIG. 3 is a schematic diagram of another example insert in accordance with the present technology;

FIG. 4 is a cross-section diagram of an example cartridge in accordance with the present technology;

FIG. 5A is a schematic diagram of another example insert in accordance with the present technology;

FIG. 5B cross-section diagram of a cartridge in accordance with the present technology;

FIG. 6 is a schematic diagram of a capped insert coupled to a catheter in accordance with another example of the present technology;

FIG. 7 is a plot of an exemplary release profile of an active agent in terms of concentration of active agents vs. the volume of aqueous solution injected through the catheter and insert;

FIG. 8 is a plot of an additional exemplary release profile of an active agent in terms of concentration of active agent vs. the volume of aqueous solution injected through the catheter and insert; and

FIG. 9 is a plot of an exemplary release profile of an active agent in terms of concentration, where the active agent is injected through a vessel that can slow down the output of the active agent.

### DETAILED DESCRIPTION

Reference will now be made to the examples illustrated, and specific language will be used herein to describe the same. Features and advantages of the technology will be apparent from the detailed description which follows, taken in conjunction with the accompanying drawings, which together illustrate, by way of example, features of the technology.

It is to be noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the description herein.

The term “catheter” is used herein to refer generally to devices used to provide fluid access to or from internal body spaces of a subject. This includes transcutaneous access as well as access through ducts, tracts, or passages of the subject's body. These access devices include, without limitation, vascular catheters, venous catheters, arterial catheters, feeding tubes, injection ports, perfusion ports, urinary catheters, ventricular shunt ports, and the like.

The terms “connector,” “fitting,” “luer-type connector” or the like refer to the connection portion of two or more volumes at the location where they are joined together. For example, one type of connector is commonly referred to as a luer connector, a luer-type connection, or a luer fitting. These connectors typically include female luer fitting and a male luer fitting, e.g., female luer fitting at the end or along a catheter tube and a male luer nozzle at the end or along a fluid delivery or extraction device (or vice versa). The female and male portion together comprise the “connector” or “fitting,” whether part of the catheter tube or part of the fluid delivery or extraction device. The term “luer-type” connector should not infer one type of connector and thus includes both traditional luer connectors as well as other connector configurations that may be useful, including barbed connectors, pressure fit connectors, threaded connectors, etc. In some examples, the connector need not be for a catheter, but can be for a fluid directing device, e.g., needle, tube, etc., that is used to supply fluid to a container or substrate.

The term “active agent” is used to refer to pharmaceutically active agent(s), chemotherapeutic agents, or agents that promote, improve, or extend the viability of catheters, particularly by acting in the lumen of the catheter to prevent, retard, or ameliorate processes that compromise access or threaten patient health. Such processes include, without limitation, pathogenic invasion and infection, blood clotting, plaque buildup, and fungal growth. Active agents can also, in some applications, include drugs, such as chemotherapeutic agents or other pharmaceutical agents.

Sizes, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1.0 to 2.0 percent” should be interpreted to include not only the explicitly recited values of about 1.0 percent to about 2.0 percent, but also include individual values and sub-ranges within the indicated range. Thus, included in this numerical range are individual values such as 1.1, 1.3, and 1.5, and sub-ranges such as from 1.3 to 1.7, 1.0 to 1.5, and from 1.4 to 1.9, etc. This same principle applies to ranges reciting only one numerical value. Furthermore, such an interpretation should apply regardless of the breadth of the range or the characteristics being described.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The present disclosure is directed to methods of tuning the release of active agents from cartridges disposed within a catheter as well as related devices. In one embodiment, a method of tuning the release of an active agent from a cartridge adapted for insertion into a catheter connector can comprise steps of establishing a desired release profile for an active agent to be delivered into a catheter, and disposing a cartridge having the active agent associated therewith into a fluid pathway of a catheter connector of the catheter. Thus, when an aqueous solution contacts and passes by or through the cartridge, the active agent is released from the cartridge at the desired release profile. As a note, any aqueous solution that is effective for disassociating the active agent from the cartridge can be used, e.g., saline, alcoholic solutions that include water, etc. However, in one example, the aqueous solution used can be a physiologic saline or a solution that includes saline.

In another embodiment, a method of tuning the release of an active agent from a cartridge adapted for insertion into a catheter connector can comprise steps of determining a desired release profile for an active agent to be delivered into or through a catheter when contacted with an aqueous solution, and obtaining a cartridge configured to be disposed into a catheter connector along a fluid pathway of the catheter. Additional steps can include associating the active agent with the cartridge to provide the release profile, and disposing the cartridge into the catheter connector.

Regarding these methods, the cartridge associated with the active agent can be adapted to include one or multiple of the following properties: i) the cartridge has a porosity configured to delay or modulate the release of the active agent; ii) the active agent is chemically modified to alter its solubility in a predetermined flushing or locking solution; iii) the active agent is lyophilized on or into a surface of the cartridge; iv) a coating layer is included on the cartridge that delays or modulates the release of the active agent from the cartridge; v) the active agent is absorbed beneath a surface of the cartridge and/or vi) the active agent, in solution, is passed through activated carbon or silica or such similar agents as to slow the active agents release.

In addition to these methods, various inserts for luer-type catheter connections are disclosed herein. In one example, the insert can comprise a cartridge adapted for insertion into a fluid pathway of a catheter connector, and the cartridge can have a lyophilized active agent associated therewith. In this example, the cartridge can be configured to release the lyophilized active agent into the catheter when fluid is passed along or through the cartridge.

In another example, the insert can comprise a cartridge adapted for insertion into a fluid pathway of a catheter connector, and the cartridge can include a chemically modified active agent associated therewith, e.g., the chemically modified active agent can be modified to alter (hasten, delay, or modulate active agent release) a release profile compared to an unmodified active agent. In this example, the cartridge can be configured to release the chemically modified active agent into the catheter when fluid is passed along or through the cartridge.

In another example, the insert can comprise a cartridge adapted for insertion into a fluid pathway of a catheter connector, and the cartridge can have an active agent associated therewith along with a coating layer applied to one or both the cartridge or the active agent. For example, the coating layer can be applied over the cartridge and the active agent to physically modulate the active agent exposure to aqueous solutions and therefore release from the cartridge when or as the coating layer is dissolved therefrom. Alternatively, the modification of the active agent with the coating can be such that the active agent and coating form a complex which modifies its release from the cartridge or its activity in solution. In still another example, the coating layer can be applied to the cartridge, but can be undercoated with respect to the active agent, and the coating layer can assist in providing desired active agent release properties. In these or other examples, essentially, the coating layer can be adapted or formulated to modify release of the active agent into the catheter when fluid is passed along or through the cartridge. Modification can be by hastening or delaying drug release, or by modulation/changing the release profile curve. Coating thicknesses, depending on the application, can range from 0.01 μm to 100 μm, for example.

In another example, the insert can comprise a cartridge adapted for insertion into a fluid pathway of a catheter connector, and the cartridge can have an active agent associated therewith. In this example, the cartridge can have a porosity configured to control (e.g., hasten, modulate, or delay) release of the active agent from the cartridge and into the catheter when fluid is passed along or through the cartridge.

In another example, the insert can comprise a cartridge adapted for insertion into a fluid pathway of a catheter or fluid directing device. The cartridge can have a pharmaceutical agent associated therewith (such as a chemotherapeutic agent). The cartridge can be adapted to release pharmaceutical agent into the catheter or fluid directing device when fluid is passed along or through the cartridge. This embodiment is particularly useful for controlled release of a drug or for a bolus delivery of a drug, particularly when there may be safety concerns related to a practitioner contacting the pharmaceutical agent while caring for a subject or patient. The catheter or fluid directing device can be the catheter for delivering the pharmaceutical agent to a subject, or can be a fluid directing device that is adapted for delivery to a fluid container or substrate for subsequent oral, topical, intravenous, intramuscular, ocular, anal, vaginal, mucosal, or transdermal preparation or delivery.

In another example, a method of delivering an active agent into the lumen of a catheter or a patient can comprise flowing an aqueous solution in contact with the insert of any one of the devices described herein to cause the active agent to enter the lumen of the catheter and/or the patient.

In another example, the active agent can be solubilized and passed through a secondary connector encapsulating activated carbon or slica or similar agent that through ionic or other physical interactions slows the output of the active agent.

In each of the insert examples, the insert can include a male end configured for insertion into a female luer fitting of a catheter, and a female end configured to receive a male luer nozzle of a fluid delivery device. It is noted that the term “fluid delivery device” does not preclude fluid extraction, and should be interpreted broadly to include any fluid movement effectuated by the “fluid delivery device,” whether injecting or withdrawing fluid. The insert can also provide a fluid pathway connecting the female end and the male end; thus, the cartridge is positioned such that the fluid pathway is fluidly coupled to the cartridge. In one example, the fluid pathway is primarily between the cartridge and an interior wall of the catheter. In another example, the fluid pathway is primarily through the cartridge.

It is noted that when discussing the present methods and inserts, each of these discussions can be considered applicable to each of these embodiments, whether or not they are explicitly discussed in the context of that embodiment. Thus, for example, in discussing a method that utilizes a porosity for a cartridge for affecting the release profile of the active agent, such a porosity can also be used in a connection with the cartridges used in the inserts disclosed herein, and vice versa.

Furthermore, the mechanism of release of the active agent from the cartridge can vary from application to application. For example, the release profile may be an elution profile or curve where the active agent is separated from a material by elution as the aqueous solution is passed by or through the cartridge. However, in examples where there are coatings, for example, the release profile may be the result of initially a mechanical protection by a sugar or other coating, followed by elution of the active agent from the cartridge. In some embodiments, the active agent can be passed through an activated carbon or silica as to slow the active agent release, altering the release profile. Thus, the term “release profile” is intended to include these or other design parameters, and is more related the actual release curve and/or timing that occurs based on a specific active agent/cartridge design.

Active agent concentrations for loading into the cartridge can vary greatly, depending on the cartridge material, how the active agent is associated with the cartridge, the desired concentration to be released into the catheter or subject, and many of the other concepts described herein. However, without being limiting, loading concentrations can typically range from 0.01 wt % solution to 20 wt % solution. Likewise, without limitation, CHG in particular can be loaded at solution concentrations ranging from 0.01 wt % to 5 wt %, from 0.05 wt % to 3 wt %, or from 0.1 wt % to 1 wt %, for example. There may be embodiments where loading concentrations outside of this range may be beneficial.

The cartridges taught herein and utilized in the disclosed methods can be associated with a variety of active agents. Non-limiting examples of active agents that can be associated with the cartridges can include antimicrobial agents, antiviral agents, antifungal agents, antithrombotic agents, chemotherapeutic agents, other pharmaceutical agents, or combinations thereof. As would be appreciated by one of ordinary skill in the art, the exact agent or agents selected for association with the cartridge can be correlated to the subject receiving the catheter or the nature of the treatment or use of the catheter. In one embodiment, the active agent can be an antimicrobial agent, an antiviral agent, an antifungal agent, an antithrombotic agent, or combinations thereof. In another embodiment, the active agent can be a pharmaceutical agent, chemotherapeutic agent, or combination thereof. Other agents can be included as active agents or in conjunction with others of the above listed active agents. Non-limiting examples of such agents could include agents that promote the function of an active agent, e.g. by providing appropriate tonicity, pH, and salinity.

The active agent can be included in any form that can be held in the cartridge material while being releasable when locking the catheter. These can include solid forms such as powders, granules, crystals, and the like that are held in pore spaces within the cartridge. In one example, a suitable solid active agent can be formulated to go into solution or suspension in the presence of a locking or flushing fluid (or even a delivery fluid in the case of delivering pharmaceutical or chemotherapeutic agents), such aqueous fluids that include a physiologically appropriate saline solution. In another example, the cartridge can be impregnated with an active agent in fluid form which then dries and adheres to the cartridge material. Infusion of the cartridge with an aqueous (locking, flushing, delivery) fluid then reconstitutes the active agent for delivery into or through the catheter. The physical state of the active agent can also be modified in order to provide the desired release profile from the cartridge. For example, in one aspect, the active agent can be prepared prior to association with the cartridge, and/or can be deposited as part of a lyophilization process, or an adsorption process onto the cartridge, or an absorption process where the active agent is associated with the cartridge. Regardless, the association of the active agent with the cartridge can be accomplished through any method known in the art. For example, the active agent can be impregnated, chemically bound, statically bound, adsorbed onto or absorbed into the cartridge, dried, lyophilized, or otherwise adhered onto a surface of the cartridge, or any combination of such known methods. The amount of active agent associated with a cartridge for the insert can vary depending on the nature of the active agent and the method used to associate the active agent with the cartridge.

To illustrate, in one specific example, it is understood that MRSA (methicillin resistant staph aureus) can typically be killed using at least about 0.5 mg/liter of chlorhexidine gluconate or chlorhexidine digluconate (CHG, chlorhexidine gluconate, and chlorhexidine digluconate can be used interchangeably) in solution, and in some situations, up to about 16 mg/liter can be used, depending on the application, e.g., from 0.0005 to 0.016 mg/ml in solution. Thus, by designing a device that provides and leaves a greater concentration of CHG in the lumen of the catheter than what is minimally needed to kill MRSA, this pathogen can be effectively killed in accordance with examples of the present disclosure. Additionally, it is noted that the active agent loaded cartridge can be made to be completely safe to humans because a concentration that may be useful for killing MRSA could still be at a low enough concentration once in the blood that it has no negative side effects in the human host. Furthermore, by utilizing the technology described herein related to tuning of the release of the active agent from the cartridge, the device can be designed so that a majority of the released active agent remains in the catheter upon flushing or locking of the catheter. Furthermore, regarding the active agent per se, though a brief example utilizing CHG has been described, it is understood that other active agents can be used to flush or lock catheters, including, without limitation, silver sulfadiazine, rifampicin, minocycline, chlorhexidine diacetate (CHA), or the like.

The active agents or the form of the active agent can be modified in order to alter or modify the release profile of the active agent from the cartridge. In one aspect, the active agent can be chemically modified to alter the solubility of the agent in an aqueous (locking, flushing, delivery, etc.) solution. The change in solubility of the active agent can shift the release of the active agent earlier or delay it later as compared to an unmodified release profile. In one embodiment, the active agent can be chemically modified to reduce the solubility of the active agent. In another example, solubilizing groups can be added to increase the solubility of the active agent and cause it to become soluble or release more quickly from the cartridge. Chemical modifications can be accomplished through the addition of any hydrophilic or hydrophobic components known in the art so long as the modification does not significantly impact the function of the active agent or make the active agent incompatible with use in catheters.

The release profile of the active agent from the cartridge can also be adapted for a specifically desired release profile through the modification of the cartridge itself. For example, in one embodiment, the cartridge can be modified such that at least a portion of the cartridge and/or the active agent associated with the cartridge is coated with a coating layer that delays the release of the active agent from the cartridge following exposure of the cartridge to an aqueous solution. Typically, the coating would be over a portion or over the entire active agent, but there are some coatings that can provide enhanced or reduced attraction between the active agent and the coating (compared to the cartridge), and would likewise slow (or alternatively increase) the release of the active agent by virtue of the presence of an undercoating. Specific examples of such an undercoating would depend on the active agent and its affinity with the undercoating compared to its affinity to the uncoated cartridge. For example, an undercoating can be prepared that includes an amphipathic molecule, i.e. having one side of the molecule that is water soluble (hydrophilic) and the other side that is not very water soluble (hydrophobic), thus, providing a non-soluble side that can be attracted or attached to the cartridge while the chemistry of the active agent is bound to the hydrophilic side for modulated release, or vice versa. Other arrangements can also be implemented as would be appreciated by one skilled in the art after considering the present disclosure.

Regarding examples where an overcoating composition is used, the active agent can be associated with the cartridge and then coated with a coating layer that impedes contact between the active agent and the aqueous solution which passes through the catheter. Once the coating layer begins to dissolve, the active agent can be released more and more rapidly, for example. In this embodiment, the coating layer can be water soluble and the chemical nature of the coating layer composition as well as the thickness of the coating layer can be selected based on the desired release profile for the active agent.

Typically, the coating layer includes water soluble materials that can be solubilized when contacted by aqueous solutions that are passed through the catheter and cartridge. The exact solubility of the material of the coating layer can be varied depending on the desired release profile. A wide range of water soluble materials can be used in the coating layer including, but not limited to carbohydrates including sugars or sugar alcohols. Examples of sugars that could be used include sucrose, glucose, dextrose, maltose, fructose, and the like. Examples of sugar alcohols include sorbitol and maltitol or the like. Other coatings can include glycols (such as polysorbate 80 and other molecular weight forms of polyethylene glycol), water soluble polymers (such as non-cross linked low molecular weight vinyl alcohols and polyurethanes), hydroxyethylcellulose, ethylcellulose polymers, ethylene vinyl acetates, polyvinyl pyrrolidones, or the like. Any combination of these materials or other materials that would be suitable for coating compositions can likewise be used. In one embodiment, the coating layer can include a carbohydrate or a sugar-based coating.

The release profile of the active agent from the cartridge can alternatively be altered by modifying the porosity of the cartridge. The porosity of the cartridge can be modified by altering the materials used to form the cartridge or by altering the manufacturing techniques of the cartridge. In one embodiment, the cartridge can be made from a ceramic material, sponge, or other polymeric, porous, or fibrous material. Non-limiting examples of materials from which the cartridge can be formed can include ceramics, high-density polyethylene (e.g., 30-50 micron pores, 15-40 micron pores, etc.), cellulose fibers or other fiber materials, polyurethane, foams, and combinations thereof. The cartridge can have a bulk volume, as a measure of porosity, of about 10% to about 90%. In a more particular embodiment, the cartridge can have a bulk volume of about 25% to about 75%.

As alluded to previously, the release profile of the active agent from the cartridge can be varied by both modifications to the active agent, as well as modifications to the cartridge itself. For example, in one embodiment, the porosity of the cartridge can be modified and the active agent can be lyophilized. In another embodiment, the active agent can be lyophilized on a surface of the cartridge and at least a portion of the cartridge can be coated with a coating layer that delays the release of the active agent from the cartridge following exposure of the cartridge to an aqueous solution. Regardless of the nature of the cartridge material or the active agent, one or more of the following can be true of the inserts of the present disclosure. First, optionally, the cartridge can include an axial channel extending axially there through. Additionally, or alternatively, the active agent, regardless of form, can be deposited on or absorbed within the cartridge.

In each of these examples where the cartridge and/or the active agent is modified to modulate, hasten (accelerate), or delay the release of the active agent from cartridge and into the catheter, the timing of release of the active agent from the cartridge can be measured with respect to the volume of fluid passed primarily through the cartridge. These measurement techniques are not included to describe how the devices and methods are to be used, but rather, to merely describe a simple technique to determine whether the device has a predetermined release profile under a given set of conditions. In other words, a device having the release profiles described below can be used with other fluid volumes, other catheter fluids, etc. In one example, a “desired release profile” may be measured such that at least 80 wt % of the active agent content associated with the cartridge is delivered from the cartridge within the first 20 wt % of a predetermined volume. In another example, the desired release profile may be such that less than 20 wt % of the active agent content associated with the cartridge is delivered from the cartridge within the first 80 wt % of a predetermined volume. In another example, the desired release profile may be such that at least 90 wt % of the active agent content associated with the cartridge is delivered from the cartridge based on a total volume representing a catheter luminal fixed volume downstream from the cartridge. In another example, the desired release profile may be such that a volume of a flush through a lumen is greater than a fixed volume of the lumen and at least 90% of the agent is released from the cartridge within the last 10% of the flush volume lumen. In another example, the desired release profile may be such that when 20 mL of the aqueous solution is passed along or through the cartridge within the lumen of a 10 French diameter catheter, no more than 5 wt % of the active agent is released in the first 18 mL of the aqueous solution. In another example, the desired release profile may be such that when 20 mL of the aqueous solution is passed along or through the cartridge within the lumen of a 10 French diameter catheter, no more than 5 wt % of the active agent is released in the first 15 mL of the aqueous solution. In another example, the desired release profile may be such that when 20 mL of the aqueous solution is passed along or through the cartridge within the lumen of a 10 French diameter catheter, no more than 10 wt % of the active agent is released in the first 15 mL of the aqueous solution. In another example, the desired release profile may be such that when 20 mL of the aqueous solution is passed along or through the cartridge within the lumen of a 10 French diameter catheter, no more than 5 wt % of the active agent is released in the first 2 mL of the aqueous solution. In another example, the desired release profile may be such that when 3.0 mL of the aqueous solution is passed along or through the cartridge within the lumen of a 10 French diameter catheter, at least 95 wt % of the active agent is released in the last 2 mL of the aqueous solution.

Notably, the 20 mL volume or the 3 mL volume above are provided merely as examples in determining release profiles as they relate to shifting release profile curves. Release profiles can be determined with this methodology, but can be used in virtually any size catheter. This is because there are many different catheter volumes as it relates to lumen diameter and catheter length. For example, a 10 French catheter may vary in length from about 15 cm to about 35 cm, or at lengths outside of this range. Thus, the design for a locking volume can be significantly different from catheter to catheter. With this in mind, what is notable is that when flushing a catheter with whatever volume is desired, a cartridge/insert can be configured so that 80%, 90%, 95%, or more of the active agent should be present in the lumen volume at the end of the flush, or in the case of locking, you may want 80%, 90%, 95%, or more of the active agent present at the beginning of the locking process. Thus, a practitioner may want 1 to 4 mL to be released at the end of a flush, or alternatively, may want 1 to 4 mL released at the beginning of a lock. Alternatively, a device could likewise be designed so that the release occurs throughout the locking or flushing with a more even release curve. With respect to a flush, by causing the active agent to be released at the end of the flush, the amount of active agent entering the human patient can be limited. Alternatively, by limiting solution passing over the cartridge during a lock, this can likewise limit undue exposure of the active agent to the patient when using active agents designed to increase life or patency of the catheter.

The inserts disclosed herein can be configured for insertion into the access end of an access device, such as a catheter, so as to communicate with the lumen of the access device. More particularly, the insert is designed to be interposed at a connection between the access device and a locking, flushing, or delivery fluid device, such as a needleless syringe or similar volume device. As described above, the insert can be further configured to hold an active agent in the cartridge and to provide a predetermined release profile of the agent into the lumen of the catheter when an aqueous fluid is passed alone or through the cartridge.

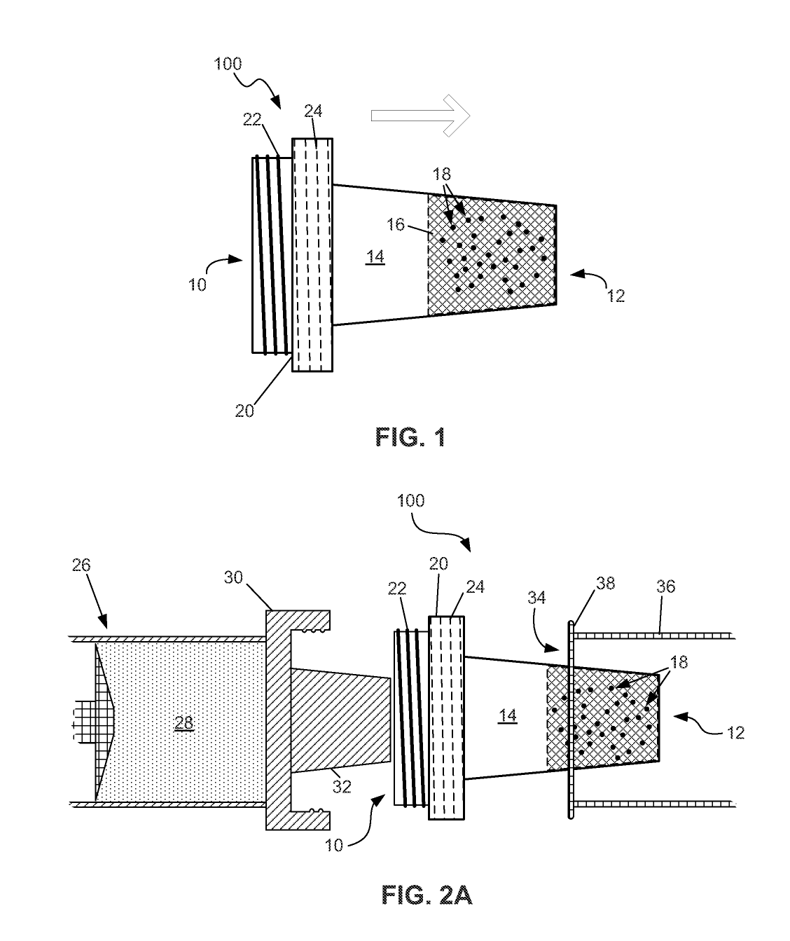

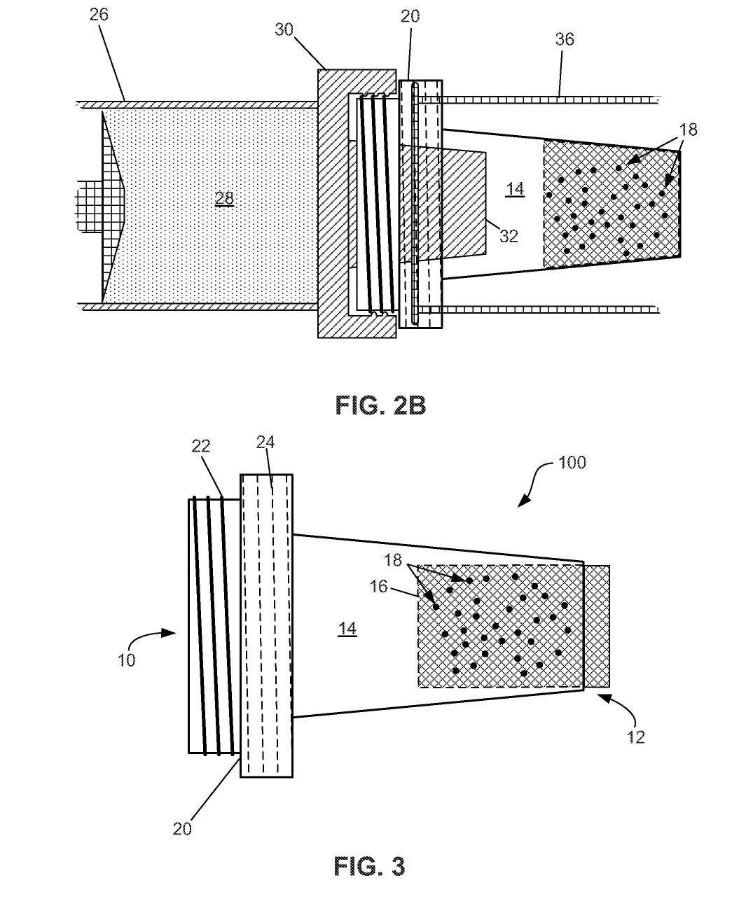

The features and function of the insert, cartridge and related methods are described above and exemplary embodiments of the insert are shown in the accompanying figures and description, such as the example shown in FIG. 1. It should be noted that FIG. 1 and subsequent FIGS. 2-6 are schematics illustrating structural features of the devices described, and are not intended to convey scale. Furthermore, the exemplified drawings are but one example of how the devices can be designed. Those skilled in the art can modify the structure shown after considering the present disclosure. With this in mind, the insert 100 has a female end 10 and a male end 12 connected by a fluid pathway 14 extending through the insert. The fluid pathway 14 can be primarily around the insert in some examples (not shown) where a wall of the catheter or catheter connector co-defines the pathway. A cartridge 16, located in the fluid pathway, is configured for admitting fluid flow, and also has an active agent 18 associated therewith, which can be released when contacted by an aqueous solution that passes through the fluid pathway and cartridge. For example, in a process of locking a catheter, an aqueous fluid can be delivered into the lumen of the catheter through the insert, where the direction of flow is indicated in FIG. 1 by an arrow. The active agent, such as chlorhexidine gluconate (CHG), in the cartridge can be released into the locking fluid according to a predetermined release profile and can enter the catheter with the fluid.

To facilitate the imposition of the insert in a luer connection, the male end of the insert is configured for insertion into a female luer fitting at the access end of a catheter. The female end of the insert can be configured to receive insertion of a male luer feature of a fluid delivery device, such as the nozzle of a syringe or tube. This male/female relationship with respect to the fluid delivery device and the catheter is fairly standard in the industry, but an alternative male/female relationship can likewise we be used without departing from the scope of the present disclosure. In an embodiment, the insert can further include features for participation in a luer-type connection. Referring again to the example in FIG. 1, a threaded collar 20 concentrically situated on the insert 100 can include threading of a size and pitch to interact with luer-type features on the connected devices. One such feature typically associated with male components of luer-type connection is a locking collar that engages with flanges or other protuberances on a female component. As shown, the threaded collar can include external threading 22 facing the female end 10 of the insert and situated to facilitate coupling of the insert with a fluid delivery device. Imposition of the insert in a luer-type connection is further facilitated by internal threading 24 facing the male end 10 of the insert and configured to engage a flange on the female luer fitting of the catheter or other access device.

The action of the collar in connecting the insert 100 is illustrated by the example shown in FIGS. 2A and 2B, in which the fluid delivery device is a syringe 26 filled with a locking fluid 28. As mentioned, the fluid delivery device need not necessarily be a device for delivering fluid, as it could likewise be used for withdrawing fluid. The term “fluid delivery device” is used primarily for convenience in describing a typical delivery action, and thus, should not be considered limiting to delivery. When the syringe is inserted in the female end 10 of the insert, the spiral external threading 22 of the threaded collar 20 engages a locking collar 30 on the syringe. Screw coupling can be achieved by rotation of the syringe or insert until the syringe nozzle 32 is securely seated in the insert. A similar principle operates for the internal threading 24 facing the male end 12 of the insert. That is, when the male end of the insert is inserted into the female luer fitting 34 of a catheter 36, the flange 38 on the catheter engages the internal threading so that relative rotation achieves coupling (a completed coupling is shown in FIG. 2B). Also shown in FIGS. 2A and 2B are fluid pathway 14 and the active agent 18 associated with the fluid pathway.

The fluid pathway inside the insert provides fluid communication with the lumen of the catheter and/or the lumen of a fluid delivery device. The dimensions of the fluid pathway from female end to male end can be selected to accommodate the luer fittings of the devices to be connected to the insert. In some cases, the dimensions of available fittings fall into one of a number of standard sizes. For example, International Organization for Standardization (ISO) specification 594 sets forth requirements for luer fittings for use with syringes and other medical transfusion apparatus. Under such standards, female fittings are larger than male fittings so as to provide an inner diameter that accommodates the outer diameter of the male fittings. One aspect of such fittings is a conical shape designed to provide a snug fit between components. As such, the lumens of such fittings typically exhibit a taper from a female end (if present) and/or toward a male end (if present). ISO-594 specifies a 6% taper. In an aspect, one or more of the dimensions of the insert are selected to conform to standard luer fittings. In one embodiment, the fluid pathway of the insert exhibits a taper extending from the female end toward the male end. In a specific embodiment the taper can be about 6%.

Referring again to FIG. 1, the insert 100 includes a cartridge 16 that is situated in the fluid pathway 14 and configured to admit fluid flow through the fluid pathway. As discussed above, cartridge can be made of any material that is suitable for admitting fluid flow and further for holding active agent and then delivering the agent into the catheter lumen. The cartridge can be shaped and sized to fit inside the insert and remain in place during use. Where the cartridge is made of a resilient material, the cartridge can be sized and shaped so as to be held in place by expansive pressure. The cartridge can be secured in place by other means such as adhesive, protuberances on the inner surface of the insert, or by a mesh screen, or any other feature that does not preclude sufficient fluid flow.

The cartridge can be placed in any position relative to the ends of the insert. In a particular embodiment, the cartridge is placed in or adjacent to the male end. In one example, the cartridge is situated wholly within the insert as shown in FIG. 1. For some uses, effective locking may be realized by exposing a substantial portion of the cartridge to the lumen of the access device. Thus, the cartridge can be within the lumen of the connector (delivery device or catheter connector portion), within the delivery device, within the lumen of the catheter tube, or any combination of these locations. Accordingly, in another example of an insert 100 shown in FIG. 3, the cartridge 16 can extend past the male end 12 of the insert so that a portion of the cartridge extends into the lumen of the catheter when installed (but may still be considered to be inserted “into” the connector, i.e. inserted through the connector and structurally held in place by the connector). In one aspect, the male end of the insert serves to guide the cartridge into the female fitting of the access device and position the cartridge within the volume of locking fluid. In another aspect, a longer cartridge can be used so as to accommodate greater amounts of active agent. The size and shape of the cartridge can further be selected so as to leave an intervening space between the cartridge and the male fitting of a fluid delivery device coupled to the insert. This example is otherwise similar to that shown in FIG. 1, and includes a female end 10 and a male end 12 connected by a fluid pathway 14 extending through the insert. The cartridge is located in the fluid pathway is configured for admitting fluid flow, and also for retaining or holding the active agent 18 that is associated with the cartridge and then releasing the agent when an aqueous fluid is flowed through the fluid pathway. Again, a threaded collar 20 is concentrically situated on the insert and can include threading of a size and pitch to interact with luer-type features on the connected devices, and can include external threading 22 facing the female end of the insert and situated to facilitate coupling of the insert with a fluid delivery device. Also included is an internal threading 24 facing the male end 10 of the insert which is configured to engage a flange on the female luer fitting of the catheter or other access device.

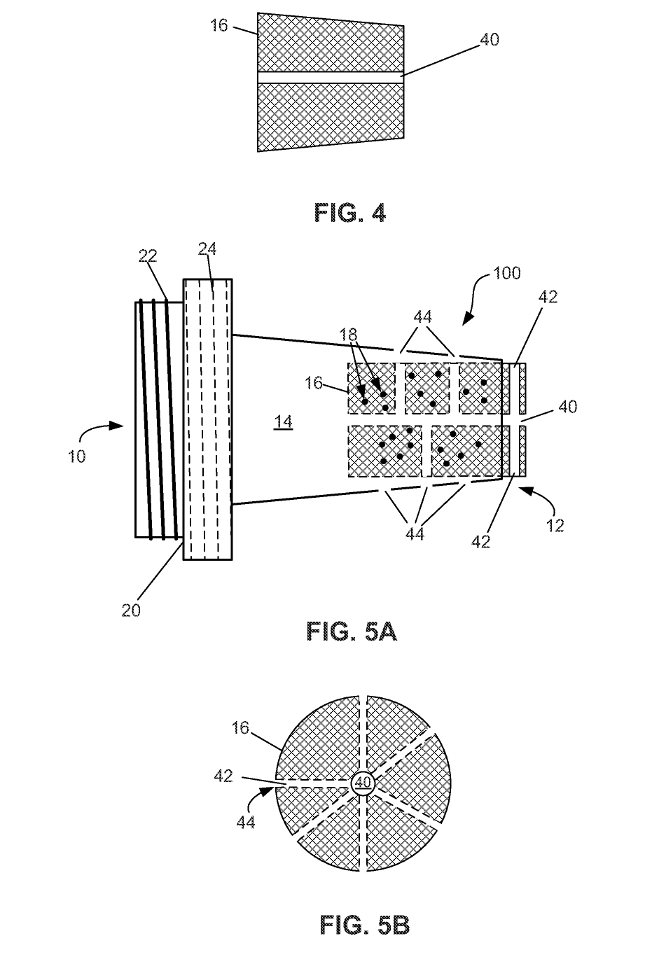

As disclosed above, the cartridge of the insert can have channels or passages which pass primarily through the cartridge in order to facilitate flow of an aqueous fluid or liquid. In a particular embodiment, an example of which is shown in FIG. 4, the cartridge 16 can include an axial channel 40 extending axially there through. Such a channel 40 can serve to decrease resistance of a cartridge to the flow of fluid through the fluid pathway and thereby provide a desired effect to the release profile of the active agent from the cartridge. In a specific embodiment, the axial channel may coincide with the center axis of the cartridge as illustrated in FIG. 4. In another aspect, the channel can serve as an interface for diffusion of active agent into the fluid. It is noteworthy that while the active agent shown in the figures, such as FIG. 4 is shown being absorbed or impregnated into the cartridge material, in some embodiments, the active agent can be disposed substantially on the surfaces of the cartridge. Such surfaces can also include internal surfaces when porous materials are used.

In a further embodiment, the axial channel 40 can communicate with the circumference of the cartridge via one or more radial channels 42 as shown in FIGS. 5A and 5B. As shown in FIG. 5A, the radial channels can be located at any point along the length of the axial channel, and can join the axial channel either singly or in groups. In a particular aspect, the radial channels extend perpendicularly with respect to the axial channel. In another aspect, as the cross-section of a cartridge shows in FIG. 5B the radial channels can radiate from the axial channel at a plurality of angles with respect to the central axis of the cartridge. By providing additional paths for fluid to flow primarily through the cartridge, the radial channels can further decrease resistance and enhance the rate of flow through the insert. In another aspect, the radial channels can aid in distribution of the fluid. Where the cartridge extends past the male end 12, radial channels in the exposed portion of the cartridge can help to redistribute and backfill the infused fluid into the more proximal portions of the access device lumen.

Another feature for enhancing fluid flow can comprise one or more holes 44 penetrating the insert to provide fluid communication with the fluid pathway 14 and/or to expose portions of the cartridge to the lumen of the access device. In this aspect, holes can be included in conjunction with cartridges having channels as well as cartridges without channels. As shown in FIG. 5A, holes can be located adjacent a portion of the cartridge, providing an additional pathway for fluid within the cartridge to exit the insert. As also illustrated in FIG. 5A and also FIG. 5b, for inserts that include radial channels the holes can be positioned in correspondence with radial channels to complete communication between the axial channel and the lumen of an access device. In FIGS. 5a and 5b, other numerical references shown but not specifically discussed are similar to those described with respect to FIGS. 3 and 4.

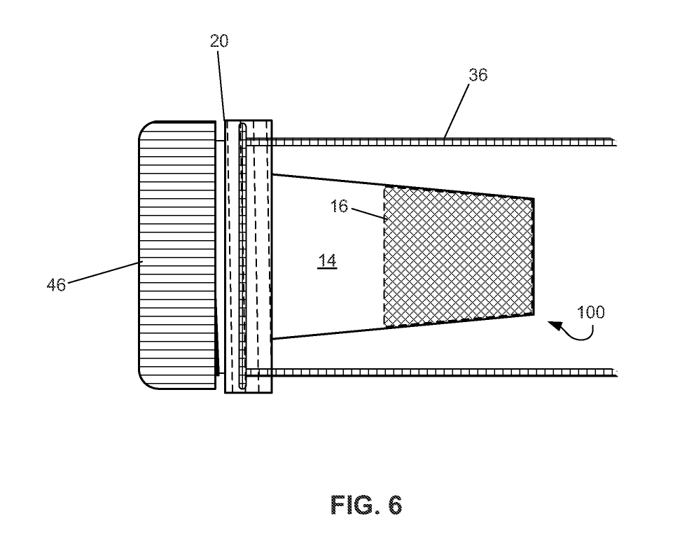

As shown in FIG. 6, the insert 100 can include an end cap 46 that can be removably attached to the female end of the insert. The end cap can serve as a barrier to seal the insert (and therefore the catheter 36) from access. Some access placements call for injectable end caps that can be penetrated by a needle and then self-seal. In a particular embodiment, the insert includes a self-sealing injectable end cap for this mode of use. Again, as shown is the cartridge 16, fluid pathway 14, and threaded collar 20.

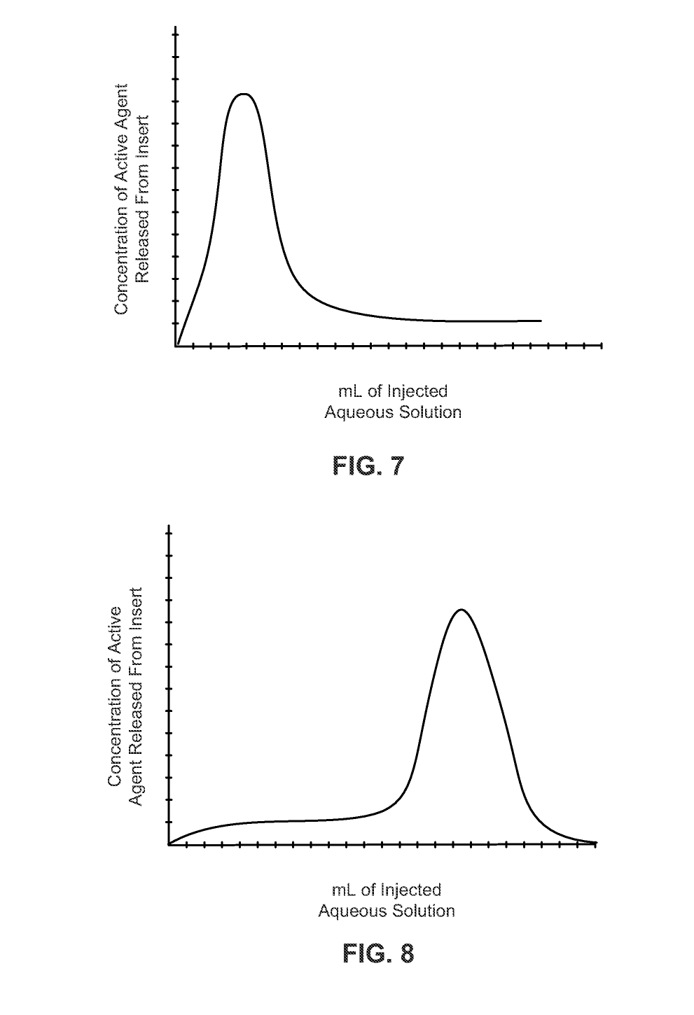

The methods and inserts disclosed herein can provide a wide range of release profiles through the unique ability to tune the release of the active agent to a desired profile. In some situations, it may be desirable to have the active agent release very quickly following the passage of a portion of an aqueous solution. An example plot of a possible release profile of active agent that occurs substantially immediately following the contact of an aqueous solution is shown in FIG. 7. Techniques for hastening release as described herein can be used to generate a release curve similar to that shown in FIG. 7, or even a faster curve in some examples. In contrast, in some aspects it may be desirable to delay the release of the active agent until a substantial volume of the aqueous solution has passed over or through the insert and cartridge. FIG. 8 shows a plot of a possible release profile in which the highest concentration of active agent delayed until a certain volume of aqueous solution has passed through or over the insert and cartridge. Again, any of the techniques described herein to slow or delay release can be used. It is noted that units are not given for the curves shown in FIGS. 7 and 8 because they will be dependent on the design of the cartridge, the active agent used, the volume of aqueous solution to be flowed over the cartridge and through the catheter, etc. However, it is understood that the X-axis generally relates to fluid volume (e.g., 5 to 50 mL) and the Y-axis generally relates active agent concentration in the fluid within the catheter, which is active agent dependent.

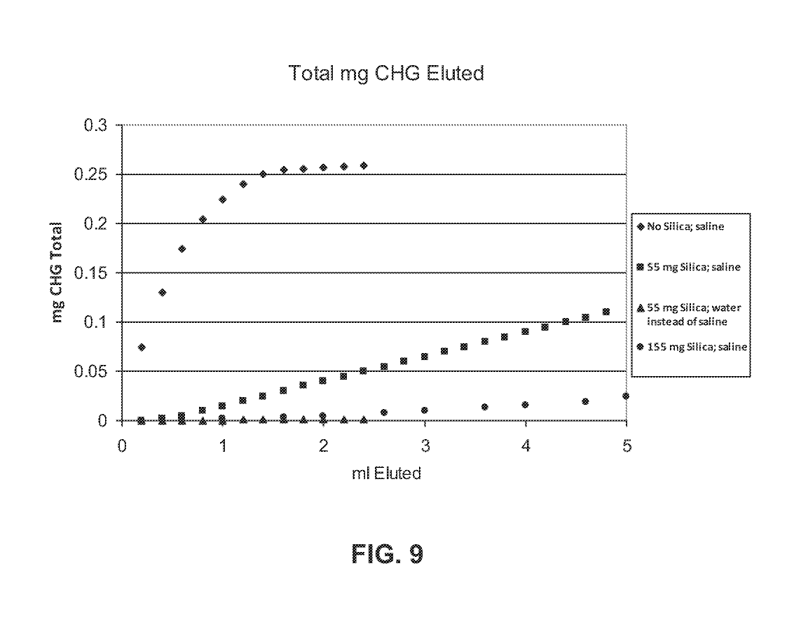

In a further embodiment, the insert can include a secondary connector that is encapsulated with activated carbon or silica or can have a surface coating with activated carbon or silica that slows the output of the active agent. The use activated carbon or silica in combination with the insert can be beneficial when controlled release of an active agent is desired for an extended period of time. FIG. 9 plots an example controlled release showing silica is usable in an insert to control the release of an active agent, namely CHG in this example.

In still another example, it may be desirable to modulate release. A wider more even release profile may be the desirable in some circumstances. While not expressly shown, it should be noted that the release peak of the profile of the active agent can be shifted forward and backward, or can be designed to modulate release with respect to the volume of aqueous solution passed over or through the insert and cartridge.

### Example

Three sample cartridge materials were each loaded at three different loading concentrations of chlorhexidine gluconate (CHG) in solution (for a total of nine samples). The three cartridge materials selected were high density polyethylene 30-50 micron from Porex Corporation, high density polyethylene 15-40 micron from Porex Corporation, and cellulose fiber (0.4 g/mL density) from Porex Corporation. Three millimeter lengths of each was cut into three samples, and each of the three samples was loaded with 0.01 wt % CHG, 0.1 wt % CHG, and 1 wt % CHG, respectively, with the balance being deionized water.

It is noted that higher active agent concentrations typically provide greater active agent concentrations within the lumen of the catheter upon flowing of a physiologic saline (or other appropriate fluid) across the cartridges. Porosity and/or density also impact the release rate of the active agent from the cartridge. Generally, lower porosity leads to higher concentrations being released more quickly for a given load density. Higher porosity generally leads to a more delayed release for a given load density. However, it is noted that other factors can also come into play that can impact the release profile, such as affinity between the active agent and the cartridge, whether there a coating or other chemical modification, lyophilization, etc.

While the forgoing example and description is illustrative of the principles of the present technology in one or more particular applications, it will be apparent to those of ordinary skill in the art that numerous modifications in form, usage and details of implementation can be made without the exercise of inventive faculty, and without departing from the principles and concepts of this technology. Accordingly, it is not intended that the technology be unduly limited.

What is claimed is:

1. A method of tuning the release of an active agent from a cartridge adapted for insertion into a catheter connector, comprising:
establishing a desired release profile for an active agent to be delivered into a catheter; and
disposing a cartridge having the active agent associated therewith into a fluid pathway of a catheter connector of the catheter, wherein when an aqueous solution contacts and passes by the cartridge, the active agent is released from the cartridge at the desired release profile.

2. The method of claim 1, wherein the active agent includes an antimicrobial agent, an antiviral agent, an antifungal agent, an antithrombotic agent, or combinations thereof.

3. The method of claim 1, wherein the active agent is a pharmaceutical agent.

4. The method of claim 3, wherein the pharmaceutical agent is a chemotherapeutic agent.

5. The method of claim 1, wherein the active agent is chemically modified to alter the solubility of the active agent in order to provide the desired release profile of the active agent from the cartridge.

6. The method of claim 5, wherein the active agent is chemically modified to reduce the solubility of the active agent.

7. The method of claim 5, wherein the active agent is chemically modified to increase the solubility of the active agent.

8. The method of claim 1, wherein the active agent is associated with the cartridge by a lyophilization process

9. The method of claim 1, wherein the active agent is associated with the cartridge such that at least a portion of the active agent is coated with a coating layer that delays the release of the active agent from the cartridge following exposure of the cartridge to the aqueous solution.

10. The method of claim 9, wherein the coating layer is a water soluble sugar-based coating.

11. The method of claim 9, wherein the coating layer includes a carbohydrate.

12. The method of claim 9, wherein the coating layer includes a sugar or a sugar alcohol.

13. The method of claim 9, wherein the coating layer includes a glycol or a water soluble polymer.

14. The method of claim 9, wherein the thickness of the coating layer affects the delay in release of the active agent, and wherein the delay in release is measured with respect to the volume of fluid passed primarily through the cartridge.

15. The method of claim 1, wherein the active agent is associated with the cartridge such that at least a portion of the cartridge is coated with a coating layer and is further undercoated with respect to the active agent.

16. The method of claim 1, wherein porosity of the cartridge is adapted to delay release of the active agent.

17. The method of claim 1, wherein the active agent is absorbed beneath a surface of the cartridge.

18. The method of claim 1, wherein the cartridge associated with the active agent is adapted to include at least two of the following properties:
i) the cartridge has a porosity configured to delay or modulate the release of the active agent;
ii) the active agent is chemically modified to alter its solubility in a predetermined flushing or locking solution;
iii) the active agent is lyophilized on or into a surface of the cartridge;
iv) a coating layer is included on the cartridge that delays or modulates the release of the active agent from the cartridge;
v) the active agent is absorbed beneath a surface of the cartridge; and
vi) the active agent is in solution and is passed through an agent that slows the release of the active agent.

19. The method of claim 18, wherein the agent that slows the release of the active agent is activated carbon or silica.

20. The method of claim 1, wherein the cartridge is adapted to be positioned primarily within a lumen of the catheter connector.

21. The method of claim 1, wherein the active agent is chlorhexidine gluconate or derivative thereof.

22. An insert for a luer-type catheter connection, comprising a cartridge adapted for insertion into a fluid pathway of a catheter connector, the cartridge having a lyophilized active agent associated therewith, wherein the cartridge is configured to release the lyophilized active agent into the catheter when fluid is passed along or through the cartridge.

23. The insert of claim 22, further comprising:
a male end configured for insertion into a female luer fitting of a catheter;
a female end configured to receive a male luer nozzle of a fluid delivery device; and
a fluid pathway connecting the female end and the male end,
wherein the cartridge is positioned such that the fluid pathway is fluidly coupled to the cartridge.

24. The insert of claim 23, wherein the fluid pathway is primarily between the cartridge and an interior wall of the catheter.

25. The insert of claim 23, wherein the fluid pathway is primarily through the cartridge.

26. The insert of claim 22, wherein the active agent is deposited on a surface of the cartridge.

27. The insert of claim 22, wherein the active agent is absorbed beneath a surface of the cartridge.

28. The insert of claim 22, wherein the active agent includes an antimicrobial agent, an antiviral agent, an antifungal agent, a chemotherapeutic agent, an antithrombotic agent, or combinations thereof.

29. The insert of claim 22, wherein the active agent is a pharmaceutical agent.

30. The insert of claim 29, wherein the pharmaceutical agent is a chemotherapeutic agent.

31. The insert of claim 22, wherein the active agent is lyophilized to a surface of the cartridge by contacting the cartridge with an active agent-containing solution, and lyophilizing the solution in the presence of the cartridge.

32. The insert of claim 22, wherein lyophilization occurs at a temperature ranging from −30° C. to −80° C., and at a vacuum pressure of 10 to 50 millitorr.

33. An insert for a luer-type catheter connection, comprising a cartridge adapted for insertion into a fluid pathway of a catheter connector, the cartridge having a chemically modified active agent associated therewith, wherein the chemically modified active agent is modified to alter a release profile compared to an unmodified active agent, and wherein the cartridge is configured to release the chemically modified active agent into the catheter when fluid is passed along or through the cartridge.

34. The insert of claim 33, further comprising:
a male end configured for insertion into a female luer fitting of a catheter;
a female end configured to receive a male luer nozzle of a fluid delivery device; and
a fluid pathway connecting the female end and the male end,
wherein the cartridge is positioned such that the fluid pathway is fluidly coupled to the cartridge.

35. The insert of claim 34, wherein the fluid pathway is primarily between the cartridge and an interior wall of the catheter.

36. The insert of claim 34, wherein the fluid pathway is primarily through the cartridge.

37. The insert of claim 33, wherein the active agent is deposited on a surface of the cartridge.

38. The insert of claim 33, wherein the active agent is absorbed beneath a surface of the cartridge.

39. The insert of claim 33, wherein the active agent includes an antimicrobial agent, an antiviral agent, an antifungal agent, a chemotherapeutic agent, an antithrombotic agent, or combination thereof.

40. The insert of claim 33, wherein the active agent is a pharmaceutical agent.

41. The insert of claim 40, wherein the pharmaceutical agent is a chemotherapeutic agent.

42. The insert of claim 33, wherein the chemically modified active agent is modified to delay release of the active agent compared to an unmodified active agent.

43. The insert of claim 33, wherein the chemically modified active agent is modified to hasten release of the active agent compared to an unmodified active agent.

44. The insert of claim 33, wherein the chemically modified active agent is modified to modulate or change the release profile compared to an unmodified active agent.

45. The insert of claim 33, wherein the active agent is chlorhexidine gluconate or derivative thereof.

46. An insert for a luer-type catheter connection, comprising a cartridge adapted for insertion into a fluid pathway of a catheter connector, the cartridge having an active agent associated therewith and a coating layer applied to one or both of the cartridge or the active agent, wherein the coating layer is adapted or formulated to control release of the active agent into the catheter when fluid is passed along or through the cartridge.

47. The insert of claim 46, further comprising:
a male end configured for insertion into a female luer fitting of a catheter;
a female end configured to receive a male luer nozzle of a fluid delivery device; and
a fluid pathway connecting the female end and the male end,
wherein the cartridge is positioned such that the fluid pathway is fluidly coupled to the cartridge.

48. The insert of claim 47, wherein the fluid pathway is primarily between the cartridge and an interior wall of the catheter.

49. The insert of claim 47, wherein the fluid pathway is primarily through the cartridge.

50. The insert of claim 46, wherein the coating layer includes a carbohydrate.

51. The insert of claim 46, wherein the coating layer includes a sugar or a sugar alcohol.

52. The insert of claim 46, wherein the coating layer includes a glycol or a water soluble polymer.

53. The insert of claim 46, wherein the coating layer is applied at a thickness from 0.01 to 100 μm.

54. The insert of claim 46, wherein the active agent is deposited on a surface of the cartridge and the coating layer is overcoated with respect to the active agent.

55. The insert of claim 46, wherein the active agent is absorbed beneath a surface of the cartridge and the coating layer is overcoated with respect to the active agent.

56. The insert of claim 46, wherein the coating layer is coated on the cartridge and is undercoated with respect to the active agent.

57. The insert of claim 46, wherein the active agent includes an antimicrobial agent, an antiviral agent, an antifungal agent, a chemotherapeutic agent, an antithrombotic agent, or combination thereof.

58. The insert of claim 46, wherein the active agent is a pharmaceutical agent.

59. The insert of claim 58, wherein the pharmaceutical agent is a chemotherapeutic agent.

60. The insert of claim 46, wherein the coating layer is adapted to delay release of the active agent compared to an uncoated cartridge.

61. The insert of claim 47, wherein the coating layer is adapted to hasten release of the active agent compared to an uncoated cartridge.

62. The insert of claim 46, wherein the coating layer is adapted to modulate or change the release profile compared to an uncoated cartridge.

63. The insert of claim 46, wherein the active agent is dichlorhexidine gluconate or derivative thereof.

64. An insert for a luer-type catheter connection, comprising a cartridge adapted for insertion into a fluid pathway of a catheter connector, the cartridge having an active agent associated therewith, wherein the cartridge has a porosity configured to control release of the active agent into the catheter when fluid is passed along or through the cartridge.

65. The insert of claim 64, further comprising:
a male end configured for insertion into a female luer fitting of a catheter;
a female end configured to receive a male luer nozzle of a fluid delivery device; and
a fluid pathway connecting the female end and the male end,
wherein the cartridge is positioned such that the fluid pathway is fluidly coupled to the cartridge.

66. The insert of claim 65, wherein the fluid pathway is primarily between the cartridge and an interior wall of the catheter.

67. The insert of claim 65, wherein the fluid pathway is primarily through the cartridge.

68. The insert of claim 64, wherein the cartridge comprises a porous polymeric, sponge, ceramic, or fibrous material.

69. The insert of claim 64, wherein the cartridge has a bulk volume of 25% to 75%.

70. The insert of claim 64, wherein the active agent is deposited on a surface of the cartridge.

71. The insert of claim 64, wherein the active agent is absorbed beneath a surface of the cartridge.

72. The insert of claim 64, wherein the active agent includes an antimicrobial agent, an antiviral agent, an antifungal agent, a chemotherapeutic agent, an antithrombotic agent, or combination thereof.

73. The insert of claim 64, wherein the active agent is a pharmaceutical agent.

74. The insert of claim 73, wherein the pharmaceutical agent is a chemotherapeutic agent.

75. The insert of claim 64, wherein the active agent is chlorhexidine gluconate or derivative thereof.

76. An insert for a luer-type connection, comprising a cartridge adapted for insertion into a fluid pathway of a catheter or fluid directing device, the cartridge having a pharmaceutical agent associated therewith, wherein the cartridge is adapted to release pharmaceutical agent into the catheter or fluid directing device when fluid is passed along or through the cartridge.

77. The insert of claim 76, further comprising:
a male end configured for insertion into a female luer fitting of a catheter or fluid directing device;
a female end configured to receive a male luer nozzle of a fluid delivery device; and
a fluid pathway connecting the female end and the male end,
wherein the cartridge is positioned such that the fluid pathway is fluidly coupled to the cartridge.

78. The insert of claim 77, wherein the fluid pathway is primarily between the cartridge and an interior wall of the catheter.

79. The insert of claim 77, wherein the fluid pathway is primarily through the cartridge.

80. The insert of claim 76, wherein the cartridge is adapted for controlled release of the pharmaceutical agent over a predetermined period of time from the cartridge.

81. The insert of claim 76, wherein the cartridge is adapted for delivering a bolus of the pharmaceutical agent from the cartridge.

82. The insert of claim 76, wherein the pharmaceutical agent is a chemotherapeutic agent.

83. The insert of claim 76, wherein the insert is adapted for safety to reduce the risk of contact between a medical practitioner and the pharmaceutical agent.

84. The insert of claim 76, wherein the catheter or fluid directing device is the catheter and is adapted for transcutaneous or direct vascular or ductal access to a subject or patient.

85. The insert of claim 76, wherein the catheter or fluid directing device is the fluid directing device and is adapted for delivery to a fluid container or substrate for subsequent oral, topical, intravenous, intramuscular, ocular, anal, vaginal, mucosal, or transdermal preparation or delivery.

86. The insert of claim 76, wherein the cartridge comprises a porous polymeric, sponge, ceramic, or fibrous material.

87. The insert of claim 76, wherein the cartridge has a bulk volume of 25% to 75%.

88. The insert of claim 76, wherein the pharmaceutical agent is deposited on a surface of the cartridge.

89. The insert of claim 76, wherein the pharmaceutical agent is absorbed beneath a surface of the cartridge.

90. A method of tuning the release of an active agent from a cartridge adapted for insertion into a catheter connector, comprising:
determining a desired release profile for an active agent to be delivered into or through a catheter when contacted with a predetermined aqueous solution;
obtaining a cartridge configured to be disposed into a catheter connector along a fluid pathway of the catheter; and
associating the active agent with the cartridge to provide the release profile upon flow of the predetermined aqueous solution in contact with the cartridge.

91. The method of claim 90, wherein the active agent includes an antimicrobial agent, an antiviral agent, an antifungal agent, an antithrombotic agent, or combination thereof.

92. The method of claim 90, wherein the active agent is a pharmaceutical agent.

93. The method of claim 92, wherein the pharmaceutical agent is a chemotherapeutic agent.

94. The method of claim 90, wherein the active agent is chemically modified to alter the solubility of the active agent in order to provide the desired release profile of the active agent from the cartridge.

95. The method of claim 90, wherein the active agent is associated with the cartridge by a lyophilization process.

96. The method of claim 0, wherein the lyophilization process is carried out by freezing the active agent in the presence of the cartridge at a temperature ranging from −30 C to −80 C, and vacuuming the cartridge 10 to 50 millitorr.

97. The method of claim 90, wherein at least a portion of one or both of the cartridge or the active agent is coated with a coating layer that is adapted to delay the release of the active agent from the cartridge following exposure of the cartridge to the aqueous solution.

98. The method of claim 90, wherein porosity of the cartridge is adapted to delay release of the active agent.

99. The method of claim 90, wherein the active agent is deposited on a surface of the cartridge.

100. The method of claim 90, wherein the active agent is absorbed beneath a surface of the cartridge.

101. The method of claim 90, wherein the cartridge associated with the active agent is adapted to include at least two of the following properties:
i) the cartridge has a porosity configured to delay or modulate the release of the active agent;
ii) the active agent is chemically modified to alter its solubility in a predetermined flushing or locking solution;
iii) the active agent is lyophilized on or into a surface of the cartridge;
iv) a coating layer is included on the cartridge that delays or modulates the release of the active agent from the cartridge;
v) the active agent is absorbed beneath a surface of the cartridge; and
vi) the active agent is in solution and is passed through an agent that slows the release of the active agent.

102. The method of claim 101, wherein the agent that slows the release of the active agent is activated carbon or silica.

103. The method of claim 90, wherein the cartridge is adapted to be positioned primarily within a lumen of the catheter connector.

104. The method of claim 90, further comprising the step of disposing the cartridge into the catheter connector.

105. The method of claim 104, further comprising the step of flowing the predetermined aqueous solution in contact with the cartridge.

106. The method of claim 105, further comprising the step of flowing the predetermined aqueous solution at a predetermined volume.

107. The method of claim 106, wherein the release profile is such that at least 80 wt % of the active agent content associated with the cartridge is delivered from the cartridge within the first 20 wt % of the predetermined volume.

108. The method of claim 106, wherein the release profile is such that less than 20 wt % of the active agent content associated with the cartridge is delivered from the cartridge within the first 80 wt % of the predetermined volume.

109. The method of claim 106, wherein the cartridge is adapted for a catheter locking application.

110. The method of claim 109, wherein the release profile is such that at least 90 wt % of the active agent content associated with the cartridge is delivered from the cartridge based on a total volume representing a catheter luminal fixed volume downstream from the cartridge.

111. The method of claim 106, wherein the cartridge is adapted for a catheter flushing application.

112. The method of claim 111, wherein the release profile is such that a volume of a flush through a lumen is greater than a fixed volume of the lumen, and wherein at least 90% of the agent is released from the cartridge within the last 10% of the flush volume lumen.

113. The method of claim 90, wherein the desired release profile is such that when 20 mL of the aqueous solution is passed along or through the cartridge within the lumen of a 10 French diameter catheter, no more than 5 wt % of the active agent is released in the first 18 mL of the aqueous solution.

114. The method of claim 90, wherein the desired release profile is such that when 20 mL of the aqueous solution is passed along or through the cartridge within the lumen of a 10 French diameter catheter, no more than 5 wt % of the active agent is released in the first 15 mL of the aqueous solution.

115. The method of claim 90, wherein the desired release profile is such that when 20 mL of the aqueous solution is passed along or through the cartridge within the lumen of a 10 French diameter catheter, no more than 10 wt % of the active agent is released in the first 15 mL of the aqueous solution.

116. The method of claim 90, wherein the desired release profile is such that when 20 mL of the aqueous solution is passed along or through the cartridge within the lumen of a 10 French diameter catheter, no more than 5 wt % of the active agent is released in the first 2 mL of the aqueous solution.

117. The method of claim 90, wherein the desired release profile is such that when 3.0 mL of the aqueous solution is passed along or through the cartridge within the lumen of a 10 French diameter catheter, at least 95 wt % of the active agent is released in the last 2 mL of the aqueous solution.

118. A method of delivering an active agent into the lumen of a catheter or a patient, comprising flowing an aqueous solution in contact with the insert of any one of claims 21-88 to cause the active agent to enter the lumen of the catheter and/or the patient.
